(19) United States
(12) Patent Application Publication
(10) Pub. No.: US 2005/0277155 A1
(43) Pub. Date: Dec. 15, 2005

(54) Method of immobilizing probes, in particular for producing biochips

(75) Inventors: Francoise Vinet, Grenoble (FR); Antoine Hoang, Grenoble (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: COMMISSARIAT A L'ENERGIE ATOMIQUE, Paris (FR)

(21) Appl. No.: 11/206,058

(22) Filed: Aug. 18, 2005

Related U.S. Application Data
(63) Continuation of application No. 10/451,072, filed on Jun. 20, 2003, now abandoned.

(30) Foreign Application Priority Data
Dec. 22, 2000 (FR) 00/16940

(51) Int. Cl. C12Q 1/68; H01L 21/00; C12M 1/34

(52) U.S. Cl. 435/6; 435/287.2; 438/1

(57) ABSTRACT

The invention relates to a solid support comprising sites intended for the immobilization of oligonucleotide probes or of proteins or of biological cells having ligands, the sites being provided with chemical species bound to the support by a chemical binding function. The chemical species have a diol group connected to the support by an alkyl chain. Application to biochips.

### FIELD OF THE INVENTION

The present invention relates to a method of immobilizing probes, particularly for producing biological chips. It permits the manufacture of biochips with oligonucleotides, the grafting of which onto a solid support is performed by means of a covalent bond.

### DESCRIPTION OF THE PRIOR ART

Biochips may be produced by the parallel synthesis of oligonucleotides directly on a solid support. In this case, a limitation of the method exists, connected with the purity of the synthesized probes. In fact, no purification is possible after synthesis. This leads to the presence on the support of truncated sequences of length (n−1) mers for an intended length of n. This technique is suitable for high density chips (greater than 1,000 probes), the probes of which have a length comprised between 6 and 20 mers. The applications envisaged in this case are sequencing and screening of single nucleotide polymorphism (SNP).

Another path is the immobilization of probes. In this case, the probes are pre-synthesized and therefore can be purified. Two types of oligonucleotide probe immobilization are available. A purified probe may be fixed to a solid support either by adsorption, or by grafting via a specific reaction between the probe and the support.

The adsorption of the probe onto the support is a passive phenomenon, since bonds of an electrostatic type set up between the negatively charged phosphodiester skeleton of the probe and the modified support bearing positive charges occur in a non-covalent manner in the immobilization of the probe. This type of immobilization is compatible with probe lengths comprised between 70 and several hundred mers.

On the contrary, in the case of grafting the probe by a specific reaction, the phenomenon is termed active, since it is due to the reactivity and the specificity between the function connected to the probe and that introduced onto the solid support. This mode of immobilization consequently sets up covalent type bonds between the deposited probe and the support used. It is compatible with probe lengths comprised between 6 and 50-60 mers.

The immobilization of oligonucleotide probes makes use of a pair of chemical functions connected to the probe and the support. This notion of a pair of chemical functions is based on the reactivity between a nucleophilic and an electrophilic substance.

Thus several possibilities of functionalization exist, that is, of the introduction of chemical functions on the probe and on the support. Thus a nucleophilic probe may be used on an electrophilic support, an electrophilic probe on a nucleophilic support, a nucleophilic probe on a likewise nucleophilic support but in the presence of a dielectrophilic segment, and an electrophilic probe on a likewise electrophilic support in the presence of a dinucleophilic segment.

Probes made nucleophilic by an amine function are the most widespread. This function is in fact stable and very accessible commercially.

Different steps are necessary for obtaining fluorescent spots corresponding to hybridons, the products of pairing between two complementary single strands of DNA.

Obtaining a functionalized support for a biochip with probe immobilization necessitates three main steps:
cleaning the support surface before presenting reactive sites,
silanization of this surface,
activation of the sites.

These steps are followed by three other steps, which will lead to the acquisition of fluorescence signals:
immobilization of probes on the sites,
hybridization of the probes,
reading.

Two paths are principally distinguished for the introduction onto the support of anchoring sites from which chemical functions will later be created. These sites are either nucleophilic or electrophilic. They are introduced during the silanization step.

By way of example, FIG. 1 shows a nucleophilic site constituted by an amine function attached to a support by a silane.

Supports at present available for biochips have electrophilic sites by the introduction of an aldehyde function. They permit the binding of probes rendered nucleophilic by an amine function.

FIG. 3 shows a diagram of obtaining an aldehyde function on a solid support starting from an amine function coupled to a dialdehyde segment.

The biochip supports presenting an aldehyde function and known at present have the following disadvantages.

They require a coupling intermediate (see FIG. 3), which reduces the final density of probe binding sites.

Their storage poses problems, the aldehyde form having relatively low stability. In fact, it has been observed that the supports have to be used immediately after opening the box containing them.

The NH₂ group initially on the support (see FIG. 3) may be converted into NH₃⁺, favoring the adsorption of non-specific compounds. Background noise of the biochips increases as a result.

### DETAILED DESCRIPTION OF THE INVENTION

In order to provide a solution for the disadvantages of the prior art, it is proposed according to the present invention to obtain an aldehyde function (permitting immobilization of a probe) by conversion of an epoxide via a diol.

A first subject of the invention consists of a solid support constituted by a substrate comprising sites intended for the immobilization of oligonucleotide probes, proteins, or biological cells having ligands, the sites being provided with chemical species bound to the substrate by a chemical binding function, characterized in that the chemical species have a diol group connected to the support by an alkyl chain.

FIG. 2 shows an electrophilic site constituted by an epoxide function attached to a support by a silane group disposed at the end of an alkyl chain. The alkyl chain is free from oxygen.

Such a support has the advantage of being able to be stored for six months without problems, the diol form being more stable than the aldehyde form.

The substrate may be of a material chosen from among glass, silicon, and plastic.

The chemical function binding the chemical species to the support is preferably a silane function.

A second subject of the invention consists of a solid support constituted by a substrate comprising active sites for the immobilization of oligonucleotide probes, proteins, or biological cells having ligands, the sites being provided with chemical species bound to the substrate by a chemical binding function, the chemical species comprising an aldehyde group intended for immobilization of the said probes, characterized in that the aldehyde group is a group obtained by oxidation of a diol carried by an alkyl chain. The alkyl chain avoids, before the oxidation step, the presence of oxygen in the link between the diol function and the binding group on the support.

The substrate may be of a material chosen from among glass, silicon and plastic, covered or not with a binding layer.

The chemical function attaching the chemical species to the support is preferably a silane function.

A third subject of the invention consists of a biochip comprising a solid support constituted by a substrate comprising active sites as defined hereinabove, oligonucleotide probes, proteins, or biological cells having ligands being immobilized on the active sites by means of a covalent bond between the aldehyde functions of the solid support and amine functions carried by the probes.

A fourth subject of the invention consists of a method of manufacture of a solid support having sites intended for immobilizing oligonucleotide probes, proteins, or biological cells having ligands, comprising the following steps:
cleaning of a surface of a substrate,
defining said sites by positioning on the cleaned surface, chemical species bound to the substrate by a chemical binding function, the chemical species comprising a precursor intended to form an aldehyde function permitting the immobilization of probes, proteins, or biological cells having ligands, characterized in that the precursor is an epoxide function and is treated to give a diol group, the epoxide precursor being disposed at the end of an alkyl chain.

The treatment to give a diol group is advantageously an acid hydrolysis of the epoxide.

The step of cleaning may consist of cleaning a surface of a substrate of a material chosen from among glass, silicon and plastic.

The chemical function for binding of chemical species to the substrate is preferably a silane function.

The method may furthermore comprise a step consisting of oxidizing the diol to obtain an aldehyde.

A fifth subject of the invention consists of a method of manufacture of a biochip, comprising:
the manufacture of a solid support having sites intended for the immobilization of oligonucleotide probes, proteins, or biological cells having ligands according to the above method,
immobilization of oligonucleotide probes, proteins, or biological cells having ligands by covalent bond between the aldehyde functions of the solid support and amine (or oxyamine) functions carried by the probes in the 5′ or 3′ position.

### BRIEF DESCRIPTION OF THE DRAWINGS

The invention will be better understood, and further advantages and details will become apparent, on reading the following description, given by way of example and without limitation, and from the accompanying drawings.

FIG. 1, already described, shows a nucleophilic site constituted by an amine function attached to a support by a silane,

FIG. 2, already described, shows an electrophilic site constituted by an epoxide function attached to a support by a silane,

FIG. 3 already described, shows a diagram of obtaining an aldehyde function on a solid support, according to the prior art.

FIG. 4 shows a diagram of obtaining an aldehyde function on a solid support, according to the invention,

FIG. 5 is a diagram showing the diol function in vicinal position,

FIG. 6 is a diagram showing the mechanism of sodium periodate complex formation with a diol,

FIG. 7 is a diagram showing the functionalization of a substrate silanized by the method of the invention.

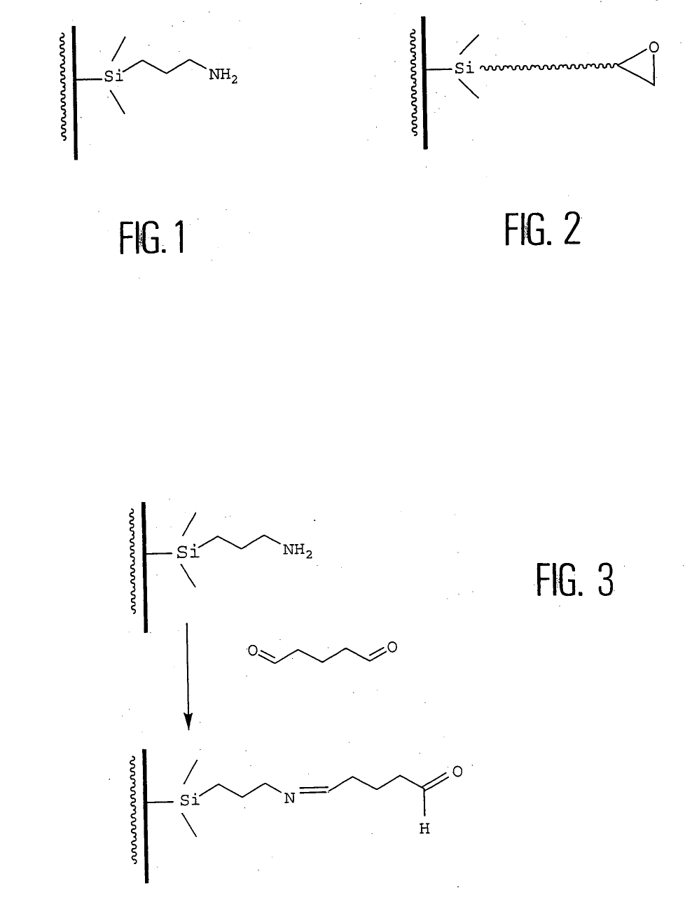

### DETAILED DESCRIPTION OF EMBODIMENTS OF THE INVENTION

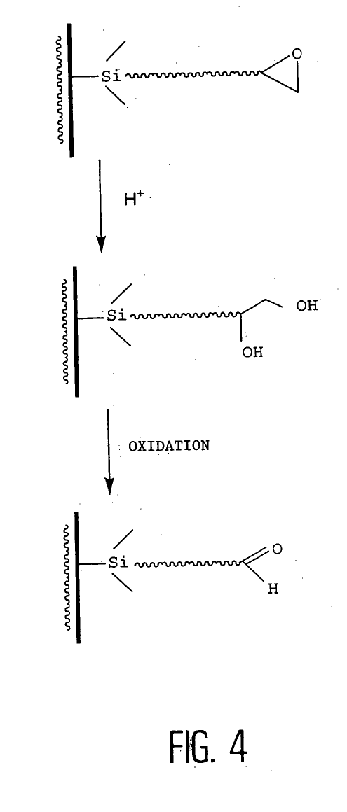

FIG. 4 is a diagram of obtaining an aldehyde function on a solid support from an epoxide function hydrolyzed to a diol, then oxidized to an aldehyde, according to the invention. The epoxide used is, for example, triethoxysilane glycidoxypropyl epoxide.

However, the inventors of the present invention have observed that, after the steps of cleaning, silanization, activation, and then hybridization, the results of fluorescence of the hybridization spots on the support are not very encouraging. This is not due to the hybridization step, since parallel manipulations performed on commercial sheets and aminated sheets gave better signal intensities.

The inventors therefore called into question, not the cleaning or silanization steps (which are effective in the case of in situ synthesis), but rather the activation step, that is, during the conversion of the epoxide function into an aldehyde and more precisely, the oxidation of the vicinal diol of the silane to an aldehyde.

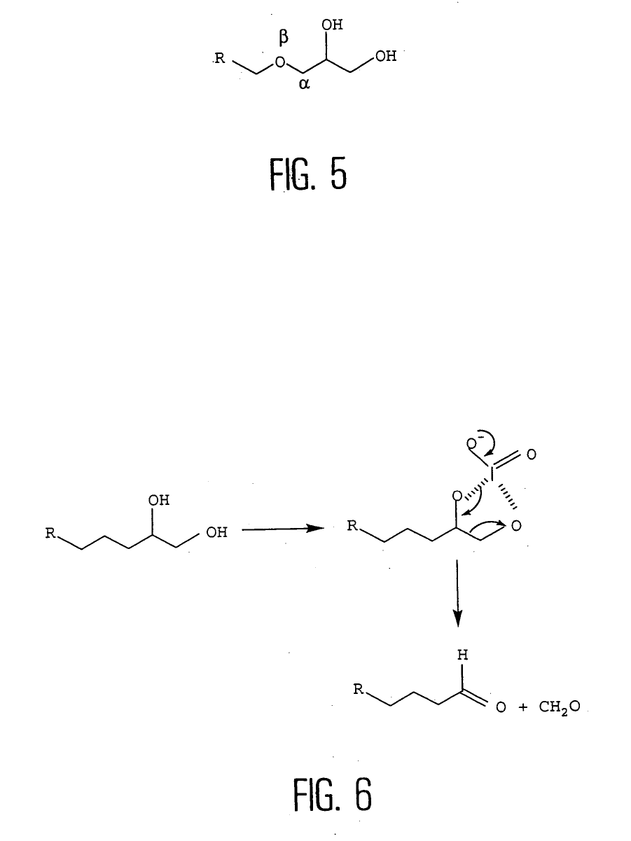

FIG. 5 is a diagram showing the diol function in vicinal position.

It follows that, with an epoxide without oxygen on the alkyl chain which carries the group, the oxidation should take place.

FIG. 6 is a diagram showing the mechanism of sodium periodate complex formation with a diol.

To verify their hypothesis, the inventors used a silane carrying an epoxide function linked by an alkyl chain (in the absence of oxygen on this chain).

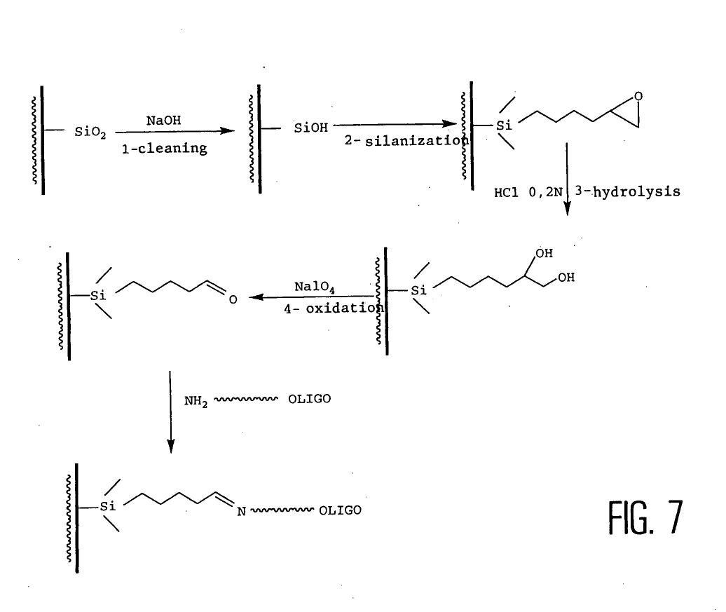

FIG. 7 is a diagram showing the functionalization of a substrate silanized by the method of the invention.

After the different steps of the reaction sequence, saturated fluorescence spots on a black background were obtained, confirming the inventors' hypothesis.

The invention has several advantages over the prior art, and more particularly over the aldehyde-type substrates at present commercially available.

The invention does not use any coupling intermediate which reduces the final density of probe binding sites. The fluorescence signal obtained is greater in the case of the invention. A factor of 10 is observed under identical experimental conditions.

As mentioned hereinabove, the invention permits storing the solid supports in the diol form, which is particularly stable.

The invention also permits minimizing the background noise of biochips, since there are no groups permitting electrostatic interactions.

The preparation of supports according to the invention can be carried over to other methods of implementing biochips, for example, in situ synthesis.

By way of example, after opening the epoxide to give a diol, the silane according to the invention has been used to effect the synthesis in situ of oligonucleotides on a solid support. The phosphoramidate chemistry currently used for the synthesis of oligonucleotides on glass beads in automatic synthesizers of the Expedite 8909 type was employed. The synthesis protocol used is described in the document Caruthers (Sciences, October 1985, page 28) 1. It comprises the steps of coupling, acetylation, oxidation and detritylation, these steps being iterated to permit the synthesis of oligonucleotides whose sequence is determined by the base (A, T, G or C) carried by the phosphoramidite nucleotide.

The first coupling step takes place between a 2′-deoxy-5′-O-dimethoxytrityl-3′-O-(β-cyanoethyl-N,N-diisopropylamino)phosphoramidite and the diol of the support. The following couplings take place between the nucleotides having different bases. A 20-mer sequence was accomplished: 3′TTTTT ATC TCA CAC AAA TAG Cy3 5′. A Cy3 fluorescent label was introduced at the end of the synthesis, using phosphoramidite chemistry as before.

The fluorescence signals measured on an Olympus BX80 epifluorescence microscope after excitation at 550 nm show an increase, greater by a factor of 4 or 5 times than those obtained in the case of a silane comprising an oxygen on the alkyl chain.

1. Solid support constituted by a substrate comprising sites intended for immobilization of oligonucleotide probes, proteins, or biological cells having ligands, the sites being provided with chemical species bound to the substrate by a chemical binding function, characterized in that the chemical species have a diol group connected to the support by an alkyl chain.

2. Solid support according to claim 1, characterized in that the substrate is a material chosen from among glass, silicon and plastic.

3. Solid support according to one of claims 1 or 2, characterized in that the said chemical function for binding the chemical species to the support is a silane function.

4. Solid support constituted by a substrate comprising sites activated for the immobilization of oligonucleotide probes, proteins, or biological cells having ligands, the sites being provided with chemical species bound to the substrate by a chemical binding function, the chemical species comprising an aldehyde group intended for immobilization of the said probes, characterized in that the aldehyde group is a group obtained by oxidation of a diol carried by an alkyl chain.

5. Solid support according to claim 4, characterized in that the substrate is a material chosen from among glass, silicon and plastic, coated or not with a binding layer.

6. Solid support according to one of claims 4 or 5, characterized in that the said chemical function for binding chemical species to the support is a silane function.

7. Biochip, characterized in that it comprises a solid support according to any one of claims 4-6, oligonucleotide probes, proteins, or biological cells having ligands being immobilized on the activated sites by means of a covalent bond between the aldehyde functions of the solid support and amine functions carried by the probes.

8. Method of manufacture of a solid support having sites intended for immobilizing oligonucleotide probes, proteins, or biological cells having ligands, comprising the following steps:
cleaning a surface of a substrate,
defining said sites by positioning on the cleaned surface chemical species bound to the substrate by a chemical binding function, the chemical species comprising a precursor intended to form an aldehyde function permitting the immobilization of probes, proteins, or biological cells having ligands, characterized in that the precursor is an epoxide function and is treated to give a diol group, the epoxide precursor being disposed at the end of an alkyl chain.

9. Method according to claim 9, characterized in that the treatment to give a diol group is a hydrolysis of the epoxide.

10. Method according to one of claims 8 or 9, characterized in that the cleaning step consists of cleaning a surface of a substrate of a material chosen from among glass, silicon, and plastic.

11. Method according to any one of claims 8-10, characterized in that the chemical function for binding of the chemical species to the substrate is a silane function.

12. Method according to any one of claims 8-11, characterized in that it furthermore comprises a step consisting of oxidizing the vicinal diol to obtain an aldehyde.

13. Method of manufacture of a biochip, characterized in that it consists of:
manufacturing a solid support having sites intended for the immobilization of oligonucleotide probes, proteins, or biological cells having ligands, according to the method of claim 12,
immobilizing oligonucleotide probes, proteins, or biological cells having ligands by covalent bond between the aldehyde functions of the solid support and amine functions carried by the probes, proteins, or biological cells having ligands in the 5′ or 3′ position.
